(19) United States
(12) Patent Application Publication
(10) Pub. No.: US 2004/0013626 A1
(43) Pub. Date: Jan. 22, 2004

(54) Material based on biodegradable polymers and method for preparing same

(76) Inventors: Ruxandra Gref, Plessis Robinson (FR); Gilles Ponchel, Paris (FR); Dominique Duchene, Paris (FR); Patrick Couvreur, Villebon-sur-Yvette (FR)

Correspondence Address: YOUNG & THOMPSON, 745 SOUTH 23RD STREET 2ND FLOOR, ARLINGTON, VA, 22202

(21) Appl. No.: 10/276,178

(22) PCT Filed: May 16, 2001
(86) PCT No.: PCT/FR01/01496

(30) Foreign Application Priority Data
May 16, 2000 (FR) 00/06232

(51) Int. Cl. A61K 7/06; A61K 7/11; C08G 63/48; C08G 63/91

(52) U.S. Cl. 424/70.13; 525/54.2

(57) ABSTRACT

A material with controlled chemical structure includes at least a biodegradable polymer material and a polysaccharide with linear, branched or crosslinked skeleton. The material is obtained by controlled functionalizing of at least a molecule of the biodegradable polymer or one of its derivatives by covalent grafting directly at its polymeric structure, of at least a molecule of the polysaccharide. A vector preferably in the form of particles obtained from the material and its use as biological vector are also disclosed.

The present invention concerns new materials based on biodegradable polymers and on polysaccharides, vectors deriving from these materials preferably in the form of particles, and their uses as biological vectors for active materials.

Vectorizing is an operation aimed at modulating and if possible totally controlling the distribution of a substance, by associating it with an appropriate system termed vector.

In the field of vectorizing, three principal functions need to be provided:

transporting the active material(s) in the biological liquids of the organism,

conveying the active materials to the organs to be treated, and

effecting the release of the active materials.

The general principle of vectorizing is of course also to render the distribution of the active material as independent as possible of the properties of the active substance itself and to subject it to that of the appropriate vectors selected according to the objective envisaged.

In fact, the coming into existence in vivo of the vector is conditioned by its size, its physico-chemical characteristics and, in particular, its surface properties which determine the interactions with the constituents of the living medium.

Several categories can be distinguished among the different vectors which already exist.

The first generation vectors are systems designed to release an active principle within the target aimed at. It is necessary in this case to have recourse to a particular mode of administration. These vectors of relatively large size (more than a few tens of microns) are either solid systems (micro-spheres), or hollow systems (micro-capsules), containing an active substance, for example an anti-cancer substance, in the dissolved state or dispersed in the constituent material of the systems. The materials usable are variable in nature (wax, ethyl cellulose, polylactic acid, copolymers of lactic and glycolic acids), biodegradable or not.

The second generation vectors are vectors capable, without a particular mode of administration, of conveying an active principle to the intended target. More precisely, they are vectors whose size is less than a micrometer and whose distribution in the organism is fully dependent on their unique physico-chemical properties.

To this second category belong in particular the liposome type vesicular vectors which are vectors constituted by one of more internal cavities containing an aqueous phase, nanocapsules which are vesicular vectors formed of an oily cavity surrounded by a polymeric wall, and also lipidic emulsions. There can also be distinguished the nanospheres which are constituted by a polymer matrix that can encapsulate active principles. Currently, nanospheres and also nanocapsules are grouped under the term “nanoparticles”. The active materials are generally incorporated at the nanoparticles either in the course of the process of polymerization of the monomers from which the nanoparticles derive, or by adsorption on the surface of the already formed nanoparticles, or during the manufacture of the particles from preformed polymers.

The present invention quite particularly concerns the field of vectors of the nanoparticle and microparticle type and their applications.

Different types of nanoparticles and microparticles are already proposed in the literature. Conventionally, they derive from a material obtained by direct polymerization of monomers (for example cyanoacrylates), by crosslinking, or they are prepared from preformed polymers: polylactic acid (PLA), polyglycolic acid (PGA), (ε-polycaprolactone (PCL), and their copolymers, such as, for example, polylactoglycolic acid (PLGA), etc. . . .

More recently, a new type of particles has been obtained from a material deriving from the catalytic polymerization of monomers (such as, for example, lactide or caprolactone), on the skeleton of a polysaccharide.

This type of material, however, has the principal drawback of not being able to guarantee a reproducible composition. In fact, all the hydroxyl functions present on the skeleton of the polysaccharide under consideration are capable of triggering the polymerization of the monomers. There are thus formed on the skeleton a very large number of chains of variable size deriving from the monomer, which “mask” the skeleton. This is a major drawback in the preparation of vectors suitable for certain applications (bioadhesion, “stealth”, targeting, etc.) where the endeavour is precisely to control the nature of the covering of the particles. Consequently, with this type of polymerization it is impossible to obtain good reproducibility of the synthesis, homogeneous samples, and to control the degrees of polymerization and substitution, the more so since polymerization is generally carried out in the mass (in the absence of solvent). In fact, during the synthesis of the material, the polysaccharide is often used in the form of particles dispersed in the melted monomer and polymerization is generally conducted in the presence of a catalyst. In the absence of a catalyst, the degrees of polymerization are very low.

There have also been described in U.S. Pat. No. 6,007,845 particles deriving from a material obtained by covalent coupling on a multi-functional material of the citric acid or tartaric acid type, of one of more molecules of a hydrophilic polymer such as polyethylene glycol and of one or more molecules of a hydrophobic polymer such as polylactic acid. However, the synthesis of this material has the major drawback of requiring the use of an annexed compound acting as insert between the molecules of the two types of polymer.

The first subject of the present invention is a new composite material with controlled structure deriving from the coupling of chains of biodegradable polymer directly on the skeleton of polysaccharides.

A second subject of the invention concerns a vector based on this material, preferably in the form of particles, and more preferably in the form of nanoparticles.

As a third subject, the invention also aims at the use of this vector, preferably of particles, in particular as biological carriers.

More precisely, the first aspect of the invention concerns a material with controlled chemical structure composed of at least one biodegradable polymer and of a polysaccharide with linear, branched or crosslinked skeleton, characterized in that it derives from the controlled functionalizing of at least one molecule of said biodegradable polymer or of one of its derivatives by covalent grafting, directly at its polymeric structure, of at least one molecule of said polysaccharide.

In contrast to the materials previously mentioned, the material perfected according to the present invention has the first advantage of having a controlled chemical structure and therefore of being perfectly reproducible as such. Its chemical composition is clearly identified.

Thus, the material claimed is preferably constituted to at least 90% by weight, and more preferably entirely, by a copolymer deriving from the controlled functionalizing of at least one molecule of a biodegradable polymer or of one of its derivatives by covalent grafting, directly at its polymeric structure, of at least one molecule of a polysaccharide with linear, branched or crosslinked skeleton.

According to a preferred mode of the invention, the material claimed contains no starting molecule, that is to say, of said biodegradable polymer or of said polysaccharide.

In the present instance, the material claimed is therefore different from a polymeric mixture in which the expected copolymer would be present but where there would also remain, in very variable amounts, the starting polymers. Such a polymeric mixture cannot be used as it is for preparing nanoparticles or microparticles.

In this case, the material claimed has a polydispersity less than or equal to 2 and preferably less than 1.5.

More precisely, the material claimed is obtained by coupling, directly at the molecule of the polysaccharide, one or more molecules of biodegradable polymer which are identical or different.

This covalent bond between the two types of molecule may vary in nature.

It may thus derive from the reaction between a carboxylic acid group with either an amine function to form an amide bond, or a hydroxyl function to form an ester bond.

It may also result from the reaction between an isocyanate group with an alcohol group to form a urethane type bond.

It may also derive from the reaction of a thiol function with a carboxylic group to lead to a thioester type bond.

All of these reactions are well known to an expert in the field and the execution thereof comes within his capabilities.

According to a preferred variant of the invention, the covalent bond established between the two molecules is of the ester or amide type.

More preferably, it derives from the reaction between a carboxylic function, activated if necessary, present on the biodegradable polymer and a hydroxyl or amine function present on the polysaccharide. The preferred activated functions of the acid are the ester of N-hydroxysuccinimide, the acid chloride and the imidazolide derived from carbonyl diimidazole. This reactive function, preferably carboxylic, may be either naturally present on the skeleton of the biodegradable polymer or have been introduced there previously at its skeleton, so as to permit its subsequent coupling to a polysaccharide molecule.

This activation of a function present on one of the molecules, preferably a carboxylic function on the biodegradable polymer, is of advantage especially when it is wished to prevent the manifestation of a secondary parasitic reaction, such as, for example, an intramolecular reaction. Thus, in the particular case where the polysaccharide has at its molecule two functions capable of reacting with each other, for example a hydroxyl function and a carboxylic function, the carboxylic function present on the biodegradable polymer is actuated previously so as to give preference to the kinetics of its reaction of coupling with the hydroxyl function of the polysaccharide to the detriment of those of an intramolecular reaction at the molecule of the polysaccharide.

The reproducibility and the homogeneity of the corresponding material are thus ensured.

The material according to the invention also has the advantage of possessing a satisfactory biodegradability by reason of the nature of the polymers of which it is constituted.

Within the meaning of the invention, the term “biodegradable” is understood to designate any polymer which dissolves or degrades within an acceptable period for the application for which it is intended, customarily for therapy in vivo. Generally, this period should be less than 5 years and more preferably less than one year when a corresponding physiological solution is exposed with a pH of 6 to 8 and at a temperature of between 25° C. and 37° C.

The chains of biodegradable polymers according to the invention are, or derive from, synthetic or natural biodegradable polymers.

Conventionally, the most frequently employed synthetic biodegradable polymers are the polyesters: PLA, PGA, PCL, and their copolymers, such as, for example PLGA. In fact, their biodegradability and biocompatibility have been widely established. Other synthetic polymers are also the subject of investigations. These are polyanhydrides, polyalkylcyanoacrylates, polyorthoesters, polyphosphazenes, polyamino acids, polyamidoamines, polymethylidene malonate, polysiloxane, polyesters such as polyhydroxybutyrate or polymalic acid, and also their copolymers and derivatives. Natural biodegradable polymers (proteins such as albumin or gelatin, or polysaccharides such as alginate, dextran or chitosan) may also be suitable.

In the present instance, the synthetic polymers are of quite particular interest since their bio-erosion is rapidly observed. However, these polymers are not always suited to be coupled with one or more polysaccharides, since they have almost no reactive groups, especially in the case of the biodegradable polyesters (PLA, PCL, etc.), and/or because these reactive groups exist only at the end of the chain. Consequently, the coupling of these polymers with a polysaccharide involves prior functionalizing of their chains with reactive groups while controlling the nature of the groups naturally present at the end of the chain. It is in particular the compounds thus obtained that it is intended to designate within the framework of the present invention by the term derivatives of biodegradable polymers.

Thus the biodegradable polymer preferably fulfils the general formula I:

(R₁)ₙ [biodegradable polymer](R₂)ₘ

in which:

n and m represent independently of each other either 0 or 1,

R₁ represents a C₁-C₂₀ alkyl group, a polymer different from the biodegradable polymer [for example polyethylene glycol (PEG), or a copolymer containing blocks of PEG or units of ethylene oxide, such as, for example, a Pluronic^{(R)} polymer], a protected reactive function present on the polymer (e.g. BOC—NH—), a carboxylic function, activated or not, or a hydroxyl function, and

R₂ represents a hydroxyl function or a carboxylic function, activated or not.

Especially preferred as biodegradable polymers according to the invention are the polyesters: polylactic acid (PLA), polyglycolic acid (PGA), ε-polycaprolactone PCL), and their copolymers, such as, for example, polylactoglycolic acid (PLGA), synthetic polymers such as polyanhydrides, polyalkylcyanoacrylates, polyorthoesters, polyphosphazenes, polyamides (e.g. polycaprolactame), polyamino acids, polyamidoamines, polymethylidene malonate, polyalkylene d-tartrate, polycarbonates, polysiloxane, polyesters such as polyhydroxybutyrate or polyhydroxyvalerate, or polymalic acid, as well as the copolymers of these substances and their derivatives.

The polyester is more preferably a polyester having a molecular weight below 50,000 g/mol and especially a polycaprolactone.

In addition to an advantageous feature in terms of biodegradability, the material according to the invention is of particular interest in terms of properties of bioadhesion and targeting for the particles which derive therefrom at organs and/or cells. It is in particular through the choice of the associated polysaccharide, and especially its composition and its structural organisation at the particles, that this second feature is more precisely obtained.

The polysaccharide(s) employed according to the invention are polysaccharides having a linear, branched or crosslinked structure, modified or not.

Under this definition it is intended to exclude from the field of the invention the polysaccharides having a cyclic structure, like the cyclodextrines.

What is understood by modified polysaccharide is any polysaccharide having undergone a change at its skeleton, such as, for example, the introduction of reactive functions, the grafting of chemical entities (molecules, aliphatic chain links, chains of PEG, etc.). This modification should of course concern few of the hydroxyl or amine groups present on the skeleton, so as to leave the great majority of them free in order then to permit the coupling of the biodegradable polymers. Thus, there are on the market polysaccharides modified by grafting of biotin, of fluorescent compounds, etc. Other polysaccharides grafted with hydrophilic chains (e.g. PEG) have been described in the literature.

The term crosslinked refers to polymers forming a three-dimensional network in contrast to simplified linear polymers. In the three-dimensional network, the chains are connected to one another by covalent or ionic bonds and the materials thus become insoluble.

The polysaccharides which are quite particularly suited to the invention are, or derive from, D-glucose (cellulose, starch, dextran), D-galactose, D-mannose, D-fructose (galactosan, manan, fructosan). The majority of these polysaccharides contain the elements carbon, oxygen and hydrogen. The polysaccharides according to the invention may thus also contain sulphur and/or nitrogen. Thus, hyaluronic acid (composed of N-acetyl glucosamine and glucuronic acid units), chitosan, chitin, heparin or ovomucoide contain nitrogen, while gelose, polysaccharide extracted from marine algae, contains sulphur in the form of (>CH—O—SO₃H) acid sulphate. Chondroitin-sulphuric acid simultaneously contains sulphur and nitrogen.

All these polysaccharides may be functionalized with biodegradable polymers according to the invention, in so far as they naturally possess free amine and/or alcohol functions.

According to a preferred variant of the invention, the polysaccharide has a molecular weight above or equal to 6000 g/mol.

In the particular case of dextran and amylose (C₆H₁₀O₅)ₙ, n varies between 10 and 620 and preferably between 33 and 220. In the case of hyaluronic acid, the molar mass varies between 5 10³ and 5 10⁶ g/mol, preferably between 5 10⁴ and 2 10⁶ g/mol. In the case of chitosan, the molar mass varies between 6 10³ and 6 10⁵ g/mol, preferably between 6 10³ and 15 10⁴ g/mol g/mol.

By way of example of the polysaccharides more particularly suited to the invention, the polydextroses such as dextran, chitosan, pullulan, starch, amylose, hyaluronic acid, heparin, amylopectin, cellulose, pectin, alginate, curdlan, fucan, succinoglycan, chitin, xylan, xanthan, arabinan, carragheenan, polyguluronic acid, polymannuronic acid, and their derivatives (such as, for example, dextran sulphate, amylose esters, cellulose acetate, etc.) may be mentioned.

Dextran, amylose, chitosan and hyaluronic acid and their derivatives are more particularly preferred.

The material according to the invention, in copolymer form, may include the biodegradable polymer and the polysaccharide in a mass ratio varying from 1:20 to 20:1 and preferably 2:9 to 2:1.

By way of example of the materials claimed, those composed of a dextran-polycaprolactone, amylose-polycaprolactone, hyaluronic acid-polycaprolactone or chitosan-polycaprolactone copolymer may be cited more particularly.

The copolymers constituting the material claimed may be in the form of two-block copolymers, have a comb structure or have a crosslinked structure.

The preferred nature of the skeleton is a polysaccharide, and the preferred nature of the grafts is a biodegradable polymer.

Two-block or comb copolymers may be obtained by working on the molar ratio of polysaccharide:biodegradable polymer during synthesis. Copolymers with crosslinked structure may be obtained from biodegradable polymers including at least two reactive functions.

The second aspect of the present invention concerns a method for preparing the material claimed.

More precisely, this method comprises bringing together at least one molecule of a biodegradable polymer or one of its derivatives carrying at least one reactive function F1 with at least one molecule of a polysaccharide with linear, branched or crosslinked skeleton and carrying at least one reactive function F2 capable of reacting with the function F1, under conditions favourable to the reaction between the functions F1 and F2 to establish a covalent bond between said molecules and in that said material is recovered.

In the case where the biodegradable polymer is polycaprolactone, the method of preparation claimed does not require the use of a catalyst as do the conventional methods. This specificity of the method claimed is therefore particularly advantageous in terms of innocuousness and biodegradability at the level of the resulting material.

Advantageously, it is a quantitative reaction, that is to say, at least one function F1 present on the molecules of polysaccharides reacts with a function F2 present on a molecule of biodegradable polymer.

To this end, the reaction is carried out under such conditions that the manifestation of any parasitic reaction is prevented, especially the involvement of one of the functions F1 or F2 in a reaction other than the expected coupling reaction. It is thus intended to avoid the intramolecular reactions mentioned previously.

According to a preferred variant of the invention, the reactive function present on the biodegradable polymer is an acid function or an activated acid function and the reactive function on the polysaccharide is a hydroxyl or amine function. Preferably, the polysaccharide and the biodegradable polymer or derivative are brought together in a mass ratio varying from 1:20 to 20:1.

In the particular case where the reactive function present on the biodegradable polymer is an acid function, the coupling reaction may be brought about by activation for example with dicyclohexylcarbodiimide (DCC) or carbonyldiimidazole (DCI). This esterification reaction comes within the capabilities of an expert in the field.

More preferably, the polysaccharides and biodegradable polymers fulfil the definitions proposed previously. In particular, they may derive from molecules of polysaccharides or biodegradable polymers which are natural and which have been modified so as to be functionalized in accordance with the present invention.

A third aspect of the invention concerns vectors constituted by a material according to the invention.

These vectors are preferably particles having a size ranging between 50 nm and 500 μm and preferably between 80 nm and 100 μm.

In fact, according to the preparation protocol used for preparing the particles from the material claimed, the size of the particles can be fixed.

According to a preferred mode of the invention, the particles have a size ranging between 1 and 1000 nm and are then termed nanoparticles. The particles of a size varying from 1 to several thousands of microns refer to microparticles.

The nanoparticles or microparticles claimed may be prepared according to methods already described in the literature, such as, for example, the technique of emulsion/evaporation of the solvent [R. Gurny et al. “Development of biodegradable and injectable latices for controlled release of potent drugs” Drug Dev. Ind. Pharm., vol 7, pp. 1-25 1981)]; the technique of nanoprecipitation by means of a water-miscible solvent (FR2 608 988 and EP 274 691). There are also variants of these methods. For example, the technique known as “double emulsion”, which is of interest for the encapsulation of hydrophillic active principles, consists in dissolving the latter in an aqueous phase, forming a water/oil type emulsion with an organic phase containing the polymer, then forming a water/oil/water type emulsion by means of a new aqueous phase containing a surfactant. After evaporation of the organic solvent, nanospheres or microspheres are recovered.

Within the framework of the present invention, the inventors have also perfected a particularly advantageous new method which comprises:

introducing a material according to the invention, mixed if necessary with another compound and/or an active material, in a liquid, preferably water, at a concentration below or equal to 50 mg/ml,

heating the whole, while stirring, to a temperature favourable to the melting or softening of said material so as to obtain its dispersion in the form of droplets,

cooling the whole so as to fix the structure thus obtained, and

recovering the particles.

It should be noted that this method is more particularly advantageous when the polymers and copolymers constituting the material claimed comprise as biodegradable polymer a derivative of polycaprolactone, and more preferably a derivative of polycaprolactone having a molecular weight below 5000 g/mol.

The material according to the present invention has the major advantage of possessing surfactant properties, owing to its amphiphilic nature. These properties may therefore be advantageously exploited during the preparation of particles, for example, so as to avoid the use of surfactants, systematically used in the above-mentioned methods. In fact, the latter are not always biocompatible and are difficult to eliminate at the end of the process.

Another advantage of the material according to the present invention is that it offers the possibility of modulating the properties which intervene in the method for manufacturing particles through the choice:

of the mass ratio of biodegradable polymer to polysaccharide and/or

of the molar masses of the biodegradable polymers and the polysaccharides under consideration.

It is thus possible to obtain copolymers that are hydrosoluble or insoluble in water, having hydrophilic-lipophilic balances that can vary between 2 and 18 (therefore making it possible to stabilize water/oil or oil/water emulsions.

Moreover, it is possible to take advantage, during the manufacture of particles, of the particular properties of certain polysaccharides composing said material. For example, it is known that alginates, pectins having a low degree of esterification, and xanthan gum may form gels in the presence of Ca²⁺ ions. It is therefore possible to envisage forming gels or particles by means of polysaccharide-biodegradable polymer materials under similar conditions. These particles will have the advantage, compared with those prepared solely from polysaccharides, of including hydrophobic chains in their matrix, permitting control of the degradation and improved encapsulation in active principles of a hydrophobic nature or including hydrophobic domains such as certain proteins.

Similarly, it is possible to envisage the formation of particles from two types of materials according to the present invention, such as, for example, from alginate/biodegradable polymer and chitosan/biodegradable polymer copolymers.

By way of illustration of the particles according to the invention, those constituted by a material deriving from at least one polyester molecule linked by an ester or amide type bond to at least one molecule of polysaccharide selected from dextran, chitosan, hyaluronic acid and amylose may be cited more particularly. The particles are preferably composed of a material deriving from a block of polycaprolactone or of polylactic acid linked by an ester or amide type bond to at least one molecule of polysaccharide selected from dextran, chitosan, hyaluronic acid and amylose.

With regard to the structures of particles that can be obtained from the material according to the invention and the above-mentioned methods, they may be variable. There can thus be distinguished:

a structure of the type having a hydrophobic core of biodegradable polymer (that can encapsulate active principles) and a hydrophilic ring of polysaccharide, which are obtained either by means of one of the above-mentioned methods or by adsorption of the material according to the invention on preformed particles;

a structure of the particles according to which the matrix of biodegradable polymer contains aqueous inclusions which can be obtained by a “double emulsion” method and suitable for encapsulation of the hydrophilic active principles. Depending on the mode of operation selected and the hydrophilic-lipophilic balance of the material, the polysaccharide may be arranged either exclusively at the aqueous inclusions, or at these inclusions and the surface of the particles. It may also protect the encapsulated active principles (proteins, peptides, etc.) with respect to interactions, often denaturing, with the hydrophobic biodegradable polymer and the organic solvent;

a structure of the hydrophillic core (polysaccharide) and hydrophobic ring (biodegradable polymer) type, when the particles are prepared from an oil in oil emulsion (for example, silicone oil-acetone) or water in oil in oil;

a micellar structure, obtained owing to the auto-association of a material according to the invention in an aqueous phase, and

a structure termed gel formed by crosslinking of the polysaccharides with biodegradable polymers including at least two reactive functions.

In the case of the present invention, the particles degrade preferably in a period ranging between one hour and several weeks.

The particles according to the invention may contain an active substance which may be hydrophilic, hydrophobic or amphiphilic and biologically active in nature.

As active biological materials, peptides, proteins, carbohydrates, nucleic acids, lipids, polysaccharides or mixtures thereof may be mentioned more particularly. They may also be organic or inorganic synthetic molecules which, administered in vivo to an animal or a patient, are capable of inducing a biological effect and/or exhibiting a therapeutic activity. They may thus be antigens, enzymes, hormones, receptors, peptides, vitamins, minerals and/or steroids.

By way of illustration of medicaments capable of being incorporated in these particles, anti-inflammatory compounds, anaesthetics, chemotherapeutic agents, immuno-toxins, immuno-suppressors, steroids, antibiotics, antivirals, antifungals, anti-parasitics, vaccinating substances, immuno-modulators and analgesics may be cited.

Similarly, it is possible to envisage associating with these active materials compounds intended to intervene at their release profile. For example, it is possible to add chains of PEG, or of polyester (modified or not), to the composition of the particles, and thus obtain particles termed composites. As already mentioned previously, it is also possible to mix several types of materials according to the present invention, to obtain mixed particles, with the aim of intervening at the release profile of the encapsulated materials and to obtain surface properties of the particles that are suited to the applications envisaged.

Finally, it is also possible to incorporate into the particles compounds having diagnostic purposes. These may thus be substances detectable by X-rays, fluorescence, ultrasounds, nuclear magnetic resonance or radioactivity. The particles may thus include magnetic particles, radio-opaque materials (such as, for example, air or barium) or fluorescent compounds. For example, fluorescent compounds such as rhodamine or Nile red may be encased in particles with hydrophobic core. Alternatively, gamma emitters (for example Indium or Technetium) may be incorporated therein. Hydrophilic fluorescent compounds may also be encapsulated in the particles, but with a lower yield compared with the hydrophobic compounds, owing to the lower affinity with the matrix.

Commercially available magnetic particles having controlled surface properties may also be incorporated into the matrix of the particles or attached in a covalent manner to one of their constituents.

The active material may be incorporated in these particles during their formation process or on the other hand be charged at the level of the particles once the latter are obtained.

The particles according to the invention may comprise up to 95% by weight of an active material.

The active material may thus be present in an amount varying from 0.001 to 990 mg/g of particle and preferably from 0.1 to 500 mg/g. It should be noted that in the case of the encapsulation of certain macromolecular compounds (ADN, oligonucleotides, proteins, peptides, etc) even lower charges may be sufficient.

The particles according to the invention may be administered in different ways, for example by oral, parenteral, ocular, pulmonary, nasal, vaginal, cutaneous, buccal administration, etc. Oral, non-invasive, administration, is a route of choice.

In general terms, the particles administered orally may undergo different processes: translocation (capture then passage of the digestive epithelium by the intact particles), bioadhesion (immobilisation of the particles at the surface of the mucous membrane by an adhesion mechanism) and transit. For these first two phenomena, the surface properties play a major role.

The fact that the particles according to the invention have numerous hydroxyl functions at the surface proves particularly advantageous for linking there a biologically active molecule, a molecule for targeting or that can be detected. It is thus possible to envisage functionalizing the surface of these particles so as to modify the surface properties thereof and/or to target them more specifically towards certain tissues or organs. Optionally, the particles thus functionalized may be maintained at the target by the use of a magnetic field, during medical imaging or while an active compound is released. Similarly, ligands of targeting molecule type such as receptors, lectins, antibodies or fragments thereof may be fixed to the surface of the particles. This type of functionalizing comes within the capabilities of an expert in the field.

The coupling of these ligands or molecules to the surface of the particles may be carried out in different ways. It may be done in a covalent manner by attaching the ligand to the polysaccharide covering the particles or in a non-covalent manner, that is to say, by affinity. Thus, certain lectins have been able to be attached by specific affinity to the polysaccharides located at the surface of particles according to the present invention, thus enhancing the cellular recognition properties of the particles. It may also be advantageous to graft the ligand by way of a spacer arm, to enable it to reach its target in an optimum conformation. Alternatively, the ligand may be carried by another polymer entering into the composition of the particles.

The invention also concerns the use of the vectors and preferably the particles obtained according to the invention for encapsulating one or more active materials as defined previously.

Another aspect of the invention also concerns the pharmaceutical or diagnostic compositions comprising vectors and preferably particles according to the invention, if necessary associated with at least one pharmaceutically acceptable and compatible carrier. For example, the particles may be administered in gastro-resistant capsules, or incorporated in gels, implants or tablets. They may also be prepared directly in an oil (such as Migliol^{(R)}) and this suspension administered in a capsule or injected at a precise site (tumour for example).

These particles are useful in particular as stealth vectors, that is to say, vectors capable of escaping the immune defense system of the organism and/or as bioadhesive vectors.

The examples and drawings hereinafter are provided by way of non-limiting example of the present invention.

### DRAWINGS

FIG. 1: Illustration by means of an optical microscope of R—PCL—COOH particles manufactured according to Example 13 (polymer synthesised according to Example 1).

FIG. 2: Distribution of hydrodynamic diameters of R—PCL—COOH particles.

### EXAMPLE 1

### R—PCL—COOH

Mono-functionalized PCL polymers of low molar mass (2 to 4000 g/mol) of the R—PCL—CO₂H (R=C₉H₁₉) type are obtained from 5.2 g of monomer (freshly distilled ε-caprolactone) and 0.3 g of high purity capric acid (C₉H₁₉CO₂H). The acid and ε-caprolactone were introduced into a spherical flask surmounted by a reflux condenser. After purging of the reagents, the spherical flask was introduced into an oil bath thermostatically controlled at 225° C. The reaction is continued for 3 hrs 30 min under an inert (argon) atmosphere. It was stopped by immersion of the spherical flask in an ice bath. The solid obtained was dissolved, hot, in 15 ml of THF, then precipitated at ambient temperature with cold methanol.

After three reprecipitations, the yield by weight of the reaction is 60-70%. The average molar masses by number (Mn) and by weight (Mw) were determined by steric exclusion chromatography (SEC) (eluent THF 1 ml/min, universal calibration carried out with polystyrene standards). Mn is 3420 g/mol and Mw is 4890 g/mol; the polydispersity index is therefore 1.4.

An average molar mass equal in number to 3200 g/mol was determined by titration with a 10⁻²M KOH/EtOH solution of the samples of polymers of about 100 mg dissolved in an acetone/water mixture.

Other polymers with different Rs were obtained by the same method, for example from caproic acid (R=C₆H₁₃).

### EXAMPLE 2

### HOOC—PCL—COOH

The bi-functionalized polymer HOOC—PCL—COOH was synthesised according to the mode of operation of Example 1.

The succinic acid (99.9%, Aldrich) used as primer was dried under vacuum at 110° C. for 24 hours. The monomer (ε-caprolactone) was purified by distillation over calcium hydride.

Polymerization from 0.2 g of succinic acid and 4 g of ε-caprolactone made it possible to obtain, after 3 hours' reaction, 3.2 g of polymer (yield by weight 76% after four consecutive precipitations).

Dosing of the terminal COOH groups by 10⁻² M KOH/EtOH made it possible to determine an acidity corresponding to a molar mass of 3500 g/mol.

By SEC, Mn is 4060 g/mol and Mw is 4810 g/mol, the polydispersity index is 1.2.

Other polymers of variable mass are obtained by changing the acid:monomer molar ratio.

### EXAMPLE 3

### R—PLA—COOH (R=C₉H₁₉)

The monomer (D,L-lactide) was purified by two recrystallizations in ethyl acetate, followed by sublimation. The catalyst (octanoate of tin) was purified by distillation under very high vacuum. The capric acid used as primer was purified by recrystallization in ethyl acetate, then dehydrated by azeotropic distillation with benzene.

The capric acid (0.12 g) and D,L-lactide (3.5 g) were introduced into a two-necked flask equipped with a reflux condenser connected to a vacuum/argon ramp. The spherical reaction flask was rendered inert, then 7 ml of anhydrous toluene were added through the septum. After dissolving, 0.284 g of catalyst were introduced and the reaction was started immediately by immersing the spherical flask in an oil bath at 120° C. After 4 hours, the reaction was stopped, the toluene was evaporated, and the polymer called R—PLA—COOH was dissolved in dichloromethane and precipitated with ethanol. After four consecutive precipitations, a constant acidity was obtained in the polymer, which was then dried.

The molar mass Mw determined by SEC is 22 kg/mol. Dosing of the terminal groups by 10⁻² M KOH/EtOH made it possible to determine an acidity corresponding to a molar mass of 21 kg/mol.

By varying the monomer/primer molar ratio and the reaction time, it was possible to obtain polymers having molar masses of between 10 and 50 kg/mol.

### EXAMPLE 4

### R—PCL—OH and R—PLA—OH (R=alkyl)

PCL or PLA polymers mono-functionalized at the end of the chain by an alcohol group (R—PCL—OH or R—PLA—OH) were synthesised according to the protocol of Example 3, but substituting for the acid primer an alcohol primer, for example C₇H₁₅OH.

5 g of caprolactone and 0.29 g of heptilic alcohol were heated in toluene under reflux for 2 hours under an inert atmosphere and in the presence of octanoate of tin in an equimolar amount with the primer. After two precipitations, the mass yield of the reaction is 54%. The molar mass Mw is 2100 g/mol.

Testing with KOH/EtOH did not allow traces of free acidity to be detected.

### EXAMPLE 5

### R—PEG—PLA—COOH (R=OMe)

The acid primer, polyethylene glycol having at one end of the chain a methoxy group and at the other a carboxylic acid group (MeO—PEG—COOH) (Shearwater Polymers, 5000 g/mol) was dried prior to the reaction. The lactide was purified by two recrystallizations (ethyl acetate) and by sublimation. The mass ratio of the reagents MeO—PEG—COOH:lactide was 1:9 and the molar ratio MeO—PEG—COOH:catalyst was 1:1. Polymerization was continued for 2 hours under an inert atmosphere under toluene (solvent) reflux. After evaporation of the toluene, the copolymer is purified by two consecutive precipitations. The mass Mw determined by SEC is 42 kg/mol.

### EXAMPLE 6

### R—PCL-ester of NHSI (R=alkyl)

The acid function of the R—PCL—COOH polymers (Example 1) is transformed into the activated ester by reacting it with N-hydroxy succinimide (NHSI), in the presence of dicyclohexyl carbodiimide (DCC), in a 1:2 (v:v) DMF:CH₂Cl₂ mixture. The DCC was added in a slight molar excess (1.1) with respect to the chains of R—PCL—COOH and the NHSI in excess with respect to the —COOH functions. The reagents were solubilized in a minimum volume of solvent, with slight heating. The reaction takes place at 50° C. for 24 hours under an inert atmosphere. After filtration of the urea formed (DCU), the solvents are evaporated and the DMF is entrained with ether. The polymer is washed with water and dried. According to the mass of DCU weighed at each synthesis of this type, the yield of the reaction is quantitative. The ester thus obtained is soluble in THF, acetone, chlorinated solvents, etc.

### EXAMPLE 7

### PCL-DEX

The R—PCL-ester NHSI (R=C₉H₁₉) polymer having the activated ester function (Example 6) is dissolved in DMSO, then an equal amount of dextran (Pharmacia, molar mass 40,000 g/mol) is introduced. The coupling reaction takes place during 144 hours at 70° C. under argon. The transesterification reaction takes place with release of NHSI. After evaporation of the solvents, the final product is washed with water to remove the NHSI and hydrosoluble copolymers, then with dichloromethane to extract traces of unreacted polyester.

With a yield of 40%, a Dex-PCL copolymer, of the comb type, is obtained, having a dextran (Dex) skeleton (molar mass 40000 g/mol) and lateral chain links of PCL linked by ester bridges. The copolymer is purified at the end of the reaction. Its overall composition is determined by elementary microanalysis and by NMR. The copolymer contains 33% by weight of PCL.

The same protocol was used for a dextran of lower molar mass, 6000 g/mol (Fluka).

### EXAMPLE 8

### Dex-PCL

3 g of R—PCL—COOH (Example 1) are dehydrated by azeotropic distillation, then dried under vacuum at 40-50° C., for one night, directly in the 50 ml spherical reaction flask surmounted by a reflux condenser and connected to a vacuum/argon ramp. 5 ml of dry THF are then added to the spherical flask. After the acid is dissolved, there is added to the spherical flask 0.243 g of carbonyl diimidazole (CDI) which dissolves rapidly. The inert mixture is brought to reflux of the THF. It is observed that CO₂ is given off. After 3 hours, the THF is evaporated.

1.29 g of dextran (Fluka, molar mass 6000 g/mol), previously dehydrated, are dissolved, hot, in 7 ml of anhydrous DMSO, then added to the spherical reaction flask containing the imidazolide intermediary of R—PCL—COOH acid. The reaction mixture is heated at 130° C. for 3 hours. The solution turns brownish. The DMSO is evaporated then the reaction product is dissolved in chloroform and introduced into a decanting flask. It is extracted with distilled water. The aqueous phase is in the form of an abundant stable emulsion. After evaporation of the solvent, substantially no residue is found in the organic phase. The aqueous phase is evaporated and a precipitate is thus obtained which is separated off. The polymer thus obtained is washed with ether and then dried. The molar mass determined by SEC (Table 1) is 11000 g/mol. This method, rapid and selective, with high yield (>80%), is preferred hereinafter.

By varying the dextran:R—PCL—COOH mass ratio in the synthesis of the Dex-PCL, it is possible to obtain by this method a series of Dex-PCL copolymers containing variable mass rates of Dex. These copolymers were characterized by chromatography by permeation on gel (refractometer and viscosimeter detectors, at 70° C.), by means of a ViscoGel column (GMHHR-H, Viscotek, GB), calibrated with Pullulan standards. The Dex-PCL copolymers were dissolved in dimethyl acetamide (DMAC) at concentrations of 5 mg/ml. The volumes injected were 100 μl. The eluent was DMAC containing 0.4% LiBr, at a flow rate of 0.5 ml/min. The molar masses were determined by the universal calibration method. Some examples are shown in Table 1.

TABLE 1
Characteristics of the starting dextran
and of three Dex-PCL copolymers having respectively 7, 5
or 3 chain links of PCL grafted at the dextran skeleton,
synthesised by using in the reaction mixture 5, 20 or 33%
by weight of dextran (relative to the total weight of
RPCL—COOH and dextran):

| | | Dex-PCL7 | Dex-PCL5 | Dex-PCL3 |
|---|---|---|---|---|
| | | PCL chain links | PCL chain links | PCL chain links |
| Copolymer | Dextran | per Dextran chain | per Dextran chain | per Dextran chain |
| Mw | 4985 | 19060 | 16000 | 10870 |
| Mn | 4670 | 13510 | 11650 | 9878 |
| Pd | 1.06 | 1.41 | 1.37 | 1.10 |
| IVn (dl/g) | 0.087 | 0.098 | 0.12 | 0.12 |
| Rgw (nm) | 2.47 | 3.92 | 4.07 | 3.57 |
| Dn/dc | 0.147 | 0.052 | 0.084 | 0.088 |
| (ml/g) | | | | |

Dex-PCL7 derives from bringing together dextran at a rate of 5% and PCL at a rate of 95%.
Dex-PCL5 derives from bringing together dextran at a rate of 20% and PCL at a rate of 80%.
Dex-PCL3 derives from bringing together dextran at a rate of 33% and PCL at a rate of 67%.

Dex-PCL7 derives from bringing together dextran at a rate of 5% and PCL at a rate of 95%.

Dex-PCL5 derives from bringing together dextran at a rate of 20% and PCL at a rate of 80%.

Dex-PCL3 derives from bringing together dextran at a rate of 33% and PCL at a rate of 67%.

Table 1. Characteristics of the starting dextran and of three Dex-PCL copolymers having respectively 7, 5 or 3 chain links of PCL grafted at the dextran skeleton, synthesised by using in the reaction mixture 5, 20 or 33% by weight of dextran (relative to the total weight of RPCL—COOH and dextran):

Mw: average molar mass by weight

Mn: average molar mass by number

Pd: polydispersity (=Mw/Mn)

Ivw: mean intrinsic viscosity by weight

Rgw: mean radius of gyration by weight

dn/dc: variation of the specific refractive index with the concentration.

The three copolymers have a low polydispersity and average molar masses by weight of between 11000 and 19000 g/mol.

### EXAMPLE 9

### Amylose-PCL

0.2 g of amylose (Fluka, extracted from potatoes) are dissolved in 8 ml of DMSO. A cloudy solution results, to which there is added 0.2 g of R—PCL-ester of NHSI (Example 6) dissolved in 3 ml of DMSO. This mixture is incubated at 70° C. for 144 hrs. After evaporation of the solvents, the solid is taken up with 200 ml of water and 200 ml of chloroform in a decanting flask. The intermediate phase containing the amphiphilic polymer is recovered and extracted once again, then dried. This treatment is a variant of the purification method of Example 7.

The yield by weight after the second extraction is 38% (wt).

The results of microanalysis make it possible to determine the overall composition of the amphiphilic copolymer obtained, which contains 32% by weight of PCL.

### EXAMPLE 10

### Chitosan-PCL

The chitosan-polycaprolactone copolymer is obtained according to the protocol of Example 9. The synthesis was carried out from crude chitosan (Fluka, 150000 g/mol) and the yield of copolymer obtained was 22% by weight. According to elementary microanalysis, the copolymer contains 67% by weight of PCL. It is of the comb type, with a skeleton of chitosan and lateral chain links of PCL linked predominantly by amide bonds.

### EXAMPLE 11

### HA-PCL

Hyaluronic acid (Accros, molar mass above 10⁶ g/mol) in the form of sodium carboxylate is dissolved in MilliQ water, and converted in the form of free acid by means of a cation superexchange resin, and lyophilized. The product thus obtained is fairly soluble in DMSO and makes it possible to carry out coupling with the NHSI ester of R—PCL—COOH, according to the protocol of Examples 7 and 9.

The hyaluronic acid-PCL comb-type copolymer is recovered in the aqueous phase. There is no intermediate phase. According to microanalysis, this copolymer contains 18% by weight of PCL.

### EXAMPLE 12

### R—PCL—COOH Nanoparticles

A well-defined mass of R—PCL—COOH synthesised according to Example 1 is dissolved in acetone to obtain a concentration of 20 mg/ml. A volume of water equal to twice the volume of acetone is poured drop-by-drop. The polymer spontaneously forms nanospheres having an average diameter of 210 nm (measured after the evaporation of the solvent), in the absence of a surfactant.

### EXAMPLE 13

### Dex-PCL Nanoparticles

A well-defined mass of Dex-PCL copolymer synthesised according to Example 7 is introduced into dichloromethane to obtain a concentration of 10 mg/ml. The polymer is dispersed and swelled by the solvent, but it does not dissolve. A volume of water two to twenty times greater than the volume of dichloromethane is added. A coarse emulsion is first formed, then refined by means of ultra-sounds. The amphiphilic copolymer stabilizes the emulsion, thus avoiding the need to add surfactants. After evaporation of the organic solvent, nanoparticles are obtained.

The average diameter of the particles is determined by light diffusion (PCS). The size of the particles, generally less than 300 nm by this method, depends on the concentration, on the ratio of the volumes of the two phases, aqueous and organic, on the time and on the strength of the ultrasound treatment.

### EXAMPLE 14

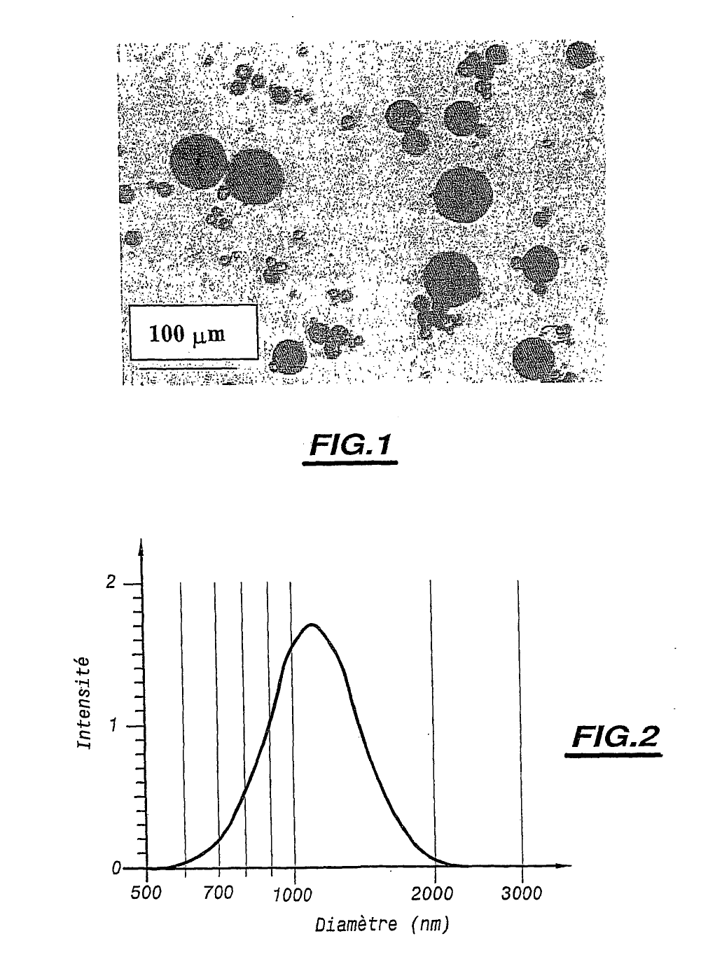

22 mg of R—PCL—COOH (Example 1) are introduced into 10 ml of MilliQ water and heated at 80° C. with magnetic stirring. Following the fusion of the polymer at this temperature, spherical particles were formed (FIG. 1). The cooling of the receiver then made it possible to fix the structures thus formed. The particles were then able to be recovered by sedimentation.

It was observed that the addition of a small amount of ethanol made it possible to improve manufacture by avoiding the formation of films at the surface of the water.

### EXAMPLE 15

Particles were formed according to the protocol of Example 14, except that instead of water a chitosan-saturated acetate buffer solution of pH 4.8 was used. Spherical particles were thus obtained.

### EXAMPLE 16

22 mg of R—PCL—COOH (Example 1) were introduced into 10 ml of MilliQ water and heated at 80° C. An ultrasound probe was then plunged into the receiver and ultrasounds were applied (20W, 20 sec.). This made it possible to obtain microspheres having an average hydrodynamic diameter of 1.1 μm (determined by PCS) and having a low polydispersity (FIG. 2).

It was noted that the use of an ultraturax could replace ultrasound treatment for the formation of the nanoparticles.

It was observed that the Dex-PCL copolymer (Example 7) and the chitosan-PCL copolymer (Example 9) also formed particles by this method.

It was noted that it was also possible to form particles by this method by replacing the water with an oil (for example Migliol^{(R)}) or with a polymer (such as PEG having a molar mass of 200 g/mol). These tests were carried out with 25 mg of polymer in 5 ml of liquid.

### EXAMPLE 17

### Bioadhesion

The interaction of the particles according to the invention with Caco2 cells in culture, used as a model of interaction for the particles intended for oral administration was studied. The tritiated PLA was encapsulated as a radioactive marker in Dex-PCL nanoparticles (Example 7) to make it possible to determine accurately the location of the particles (inside or at the surface of the cells or in the culture medium). This marking proved perfectly stable in the culture medium, therefore permitting these studies. Caco2 cells were cultured in 24-well plates, with a change of medium (1.5 ml/well DMEM 4.5 g/l glucose, 15% foetal calf serum) every 1 or 2 days to confluence. After about 4 days, when the cells have reached confluence, the medium is removed, 1.5 ml of Hank's medium are added, and after waiting for 2 hours the suspensions of nanospheres containing well-defined amounts of particles (in a total volume of 100 μl) are then added. The activity per well in the culture medium was fixed at 0.1 μCi. After three hours' incubation at 37° C. in a CO₂ incubator, the supernatant was removed, the cells were washed twice with PBS, then lysed for 1 hour with 1 ml of 0.1M NaOH. The radioactivity was counted in the supernatant, the washing waters and the cellular lysate. Thus, it was possible to determine accurately the quantity of nanoparticles effectively associated with the cells.

The quantity of Dex-PCL nanoparticles associated with the Caco2 cells is doubled compared with those of polyester (PLA, Phusis, Mw 40000 g/mol) which are manufactured by the nanoprecipitation technique (Example 10) in the presence of Pluronic^{(R)}. Thus, 2.5% and 1.1% respectively of the nanoparticles are associated with the cells.

### EXAMPLE 18

### Coupling of Lectins by Affinity, Targeting

A suspension of radio-marked nanoparticles, manufactured from Dex-PCL (Example 7), is brought into contact with a solution of lectin from peas (Lens culinaris) in excess with respect to the particles, so as to saturate the surface of the latter with lectin adsorbed by affinity. The interaction of the nanoparticles thus covered with lectin with Caco2 cells in culture was studied according to the previous protocol (Example 16).

The quantity of nanoparticles associated with the Caco2 cells is significantly increased compared with those not covered with lectin. Thus, 3.5% of the nanoparticles introduced in each well are associated with the cells, compared with 2.5% in the absence of lectin.

### EXAMPLE 19

### Stealth

The capacity of nanoparticles covered with dextran (manufactured from Dex-PCL, Example 7) to avoid capture by phagocyte cells (J774) was compared with those of the same size (approx. 200 nm) and covered with 5000 g/mol PEG (which were manufactured from PEG-PLA synthesised according to Example 4, from 5000 g/mol Me—O—PEG—OH and lactide, with a molar mass of the block of PLA of 50000 g/mol). The J774 cells were cultured in 24-well plates, in DMEM medium containing 4.5 g/l of glucose and 10% of foetal calf serum. Prior to the experiments, the supernatant of the cells was renewed, and after waiting for 4 hours the suspensions of radio-marked nanoparticles were added in the wells. The capture of the Dex-PCL nanoparticles and of the reference nanoparticles of the same size covered with PEG was substantially the same (1 to 2%), in spite of the well known capacity of this type of cells to phagocyte nanoparticles. This is an indication regarding the “stealth” character of the nanoparticles covered with dextran, similar to that of the particles covered with PEG, well known in the literature.

1. A material with controlled chemical structure composed of at least one biodegradable polymer and a polysaccharide with linear, branched or crosslinked skeleton, characterized in that it derives from the controlled functionalizing of at least one molecule of said biodegradable polymer or of one of its derivatives by covalent grafting, directly at its polymeric structure, of at least one molecule of said polysaccharide.

2. A material according to claim 1, characterized in that it is constituted by at least 90% by weight of a copolymer deriving from the controlled functionalizing of at least one molecule of a biodegradable polymer or of one of its derivatives by covalent grafting, directly at its polymeric structure, of at least one molecule of a polysaccharide with linear, branched or crosslinked skeleton.

3. A material according to claim 1 or 2, characterized in that it is free from starting product.

4. A material according to claim 1 or 2, characterized in that it has a polydispersity less than or equal to 2.

5. A material according to any one of the preceding claims, characterized in that the covalent bond established between the biodegradable polymer molecule and the polysaccharide molecule is ester or amide in nature.

6. A material according to any one of the preceding claims, characterized in that the covalent bond derives from the reaction between a hydroxyl function or an amine function present on the molecule of the polysaccharide and a carboxylic function, activated or not, present on the molecule of the biodegradable polymer.

7. A material according to any one of the preceding claims, characterized in that the biodegradable polymer fulfils the formula:
(R₁)ₙ [biodegradable polymer](R₂)ₘ
in which:
n and m represent independently of each other either 0 or 1,
R₁ represents a C₁-C₂₀ alkyl group, a polymer different from the biodegradable polymer, a protected reactive function present on the polymer, a carboxylic function, activated or not, or a hydroxyl function, and
R₂ represents a hydroxyl function or a carboxylic function, activated or not.

8. A material according to any one of the preceding claims, characterized in that the biodegradable polymer is, or derives from, a polylactic acid (PLA), polyglycolic acid (PGA), ε-polycaprolactone) (PCL), synthetic polymers such as polyanhydrides, polyalkylcyanoacrylates, polyorthoesters, polyphosphazenes, polyamides, polyamino acids, polyamido amines, polymethylidene malonate, polyalkylene d-tartrate, polycarbonates, polysiloxane, polyesters such as polyhydroxybutyrate or polyhydroxyvalerate, or polymalic acid, and also their copolymers and derivatives.

9. A material according to any one of the preceding claims, characterized in that the biodegradable polymer is a polyester having a molecular weight below 50.000 g/mol.

10. A material according to any one of the preceding claims, characterized in that the biodegradable polymer is a polycaprolactone.

11. A material according to any one of the preceding claims, characterized in that the polysaccharide has a molecular weight above or equal to 6000 g/mol.

12. A material according to any one of the preceding claims, characterized in that the polysaccharide is selected from dextran, chitosan, pullulan, starch, amylose, hyaluronic acid, heparin, amylopectin, cellulose, pectin, alginate, curdlan, fucan, succinoglycan, chitin, xylan, xanthan, arabinan, carragheenan, polyguluronic acid, polymannuronic acid, and their derivatives.

13. A material according to any one of the preceding claims, characterized in that it associates or not a biodegradable polymer and a polysaccharide in a mass ratio varying from 1:20 to 20:1 and preferably from 2:9 to 2:1.

14. A material according to any one of the preceding claims, characterized in that it is in the form of a two-block copolymer.

15. A material according to any one of the preceding claims, characterized in that it has a comb structure or a crosslinked structure.

16. A material according to any one of the preceding claims, characterized in that it is a copolymer having a polysaccharide skeleton and biodegradable polymer grafts.

17. A material according to any one of the preceding claims, characterized in that it derives from a copolymer selected from dextran-polycaprolactone, amylose-polycaprolactone, hyaluronic acid-polycaprolactone, and chitosan-polycaprolactone.

18. A method for preparing a material according to any one of the preceding claims, characterized in that at least one molecule of a biodegradable polymer or one of its derivatives carrying at least one reactive function F1 is brought together with at least one molecule of a polysaccharide with linear, branched or crosslinked skeleton and carrying at least one reactive function F2 capable of reacting with the function F1, under conditions favourable to the reaction between the functions F1 and F2 to establish a covalent bond between said molecules, and in that said material is recovered.

19. A method according to claim 18, characterized in that the biodegradable polymer is such as defined in claims 7 to 10 and the polysaccharide according to claim 11 or 12.

20. A method according to claim 18 or 19, characterized in that the reactive function of the biodegradable polymer is an activated acid function and that of the polysaccharide is a hydroxyl or amine function.

21. A method according to any one of the preceding claims, characterized in that the polysaccharide and the biodegradable polymer or derivative are brought together in a mass ratio varying from 1:20 to 20:1.

22. A vector obtained from a material according to any one of claims 1 to 17.

23. A vector according to claim 22, characterized in that it is in the form of particles.

24. A vector according to claim 22 or 23, characterized in that it is in the form of microparticles or nanoparticles.

25. A vector according to any one of claims 22 to 24, characterized in that it further comprises an active substance.

26. A vector according to claim 25, characterized in that the active substance is selected from peptides, proteins, carbohydrates, nucleic acids, lipids or organic or inorganic molecules capable of inducing a biological effect and /or with therapeutic activity.

27. A vector according to any one of claims 22 to 26, characterized in that it comprises up to 95% by weight of an active material.

28. A vector according to any one of claims 22 to 27, characterized in that it is in the form of particles further comprising at least one molecule linked in a covalent manner to its surface.

29. A vector according to any one of claims 22 to 27, characterized in that it is in the form of particles further comprising at least one molecule linked in a non-covalent manner to its surface.

30. A vector according to claim 28 or 29, characterized in that the molecule is a biologically active molecule, a molecule for targeting, or one that can be detected.

31. A vector according to claim 30, characterized in that it is a targeting molecule selected from antibodies and fragments of antibodies and lectins.

32. A vector according to any one of claims 22 to 31, characterized in that it is in the form of particles constituted by a material deriving from at least one molecule of polyester linked by an ester or amide type bond to at least one molecule of polysaccharide selected from dextran, chitosan, hyaluronic acid and amylose.

33. A vector according to any one of claims 22 to 31, characterized in that it is in the form of particles constituted by a material deriving from a block of polycaprolactone or of polylactic acid linked by an ester or amide type bond to at least one molecule of polysaccharide selected from dextran, chitosan, hyaluronic acid and amylose.

34. A method for preparing a vector in the form of particles according to any one of claims 22 to 33, characterized in that it comprises at least:
introducing a material according to any one of claims 1 to 17, if necessary with another compound and/or an active material, in a liquid, preferably water, at a concentration below or equal to 50 mg/ml,
heating the whole, while stirring, to a temperature favourable to the melting or softening of said material so as to obtain its dispersion in the form of droplets,
cooling the whole so as to fix the structure thus obtained, and
recovering said particles.

35. A method according to claim 34, characterized in that the material is a copolymer of ε-polycaprolactone having a molecular weight below 5000 g/mol.

36. A method according to claim 34 or 35, characterized in that it is carried out also in the presence of the active material to be encapsulated.

37. The use of a vector according to any one of claims 17 to 33 for encapsulating at least one active material.

38. The use according to claim 37, characterized in that the active materials are selected from peptides, proteins, carbohydrates, nucleic acids, lipids, or organic or inorganic molecules capable of inducing a biological effect and/or with therapeutic activity.

39. A pharmaceutical or diagnostic composition characterized in that it comprises as active material a vector according to any one of claims 17 to 33.

40. A diagnostic composition characterized in that it comprises as active material a vector according to any one of claims 17 to 33.

41. The use of a vector according to any one of claims 17 to 33, as “stealth” vectors.

42. The use of a vector according to any one of claims 17 to 33 as bioadhesive vectors.
